(19) United States
(12) Patent Application Publication
(10) Pub. No.: US 2024/0212800 A1
(43) Pub. Date: Jun. 27, 2024

(54) ELECTRONIC DEVICE AND METHOD FOR ASSESSING MENTAL HEALTH OF ADOLESCENT USING A SURVEY BASED ON ARTIFICIAL INTELLIGENCE

(71) Applicants: LumanLab Inc., Sejong-si (KR); Korea University Research and Business Foundation, Seoul (KR); Korea University Research and Business Foundation, Sejong Campus, Sejong-si (KR)

(72) Inventors: Jae Hyun LIM, Seoul (KR); Chul Hyun CHO, Seoul (KR); Seung Pil PACK, Sejong-si (KR)

(21) Appl. No.: 18/544,488

(22) Filed: Dec. 19, 2023

(30) Foreign Application Priority Data
Dec. 23, 2022 (KR) 10-2022-0183164

(51) Int. Cl. G16H 10/20; A61B 5/16; G16H 50/20; G16H 50/30

(52) U.S. Cl. CPC G16H 10/20; A61B 5/16; G16H 50/20; G16H 50/30; A61B 2503/06

(57) ABSTRACT

An electronic device and a method to assess a mental health state of an adolescent by using a survey formed based on an artificial intelligence (AI). The method comprises: collecting physical information of a user through a pre-installed application, verifying survey questions regarding a mental health depending on the physical information, and verifying answers to the verified survey questions through the application; forming additional survey questions after having verified the answers and calculating a prediction rate of an appearance of symptoms of a mental illness regarding the additional survey questions by using AI models; verifying a set of survey questions including a plurality of sub-questions depending on the calculated prediction rate; outputting the verified set of survey questions through the application; outputting result data by adjusting the prediction rate; and transmitting the outputted result data.

### CROSS REFERENCE TO RELATED APPLICATION

This application claims priority to Korea Patent Application No. 10-2022-0183164 filed on Dec. 23, 2022, which is hereby incorporated by reference in its entirety.

### BACKGROUND

### 1. Field of Technology

The present disclosure relates to an electronic device of a service platform system for predicting and managing the revelation of psychosomatic symptoms by periodically assessing a mental health of a school-aged adolescent through an analysis of answers to a survey based on an artificial intelligence (AI), and a method therefor.

### 2. Related Technology

Generally, a diagnosis of a mental illness of a school-aged adolescent may be confirmed through counseling with a psychiatrist and, depending on the result, a non-drug treatment or a drug treatment may be conducted.

Since the technology of communications advances and the technology of digital health care is widely used in the field of psychiatry as well, it has become possible to conduct a standardized survey regarding a certain mental illness with a mobile device or a digital apparatus that a patient has. In addition, since online telemedicine is becoming more widespread, a patient can undergo counseling with an expert through a medium such as an application or a web service.

Nevertheless, a negative social perception about psychological counseling still exists, and the fact that a direct observation by a psychiatrist in an offline medical institution is compulsory, remains as an issue. In addition, for counseling, a patient is required to have previously taken a traditional survey lasting more than 30 minutes.

Generally, a diagnosis of a mental illness of an adolescent can be started by their guardian paying a visit to an offline medical institution and taking part in a survey regarding a specific mental illness in order to obtain a verification of a psychiatrist regarding the mental health state of the adolescent.

In such a case, there are problems in that the guardian needs to arrange a meeting with a psychiatrist in order to visit a medical institution and needs to take part in various surveys. In addition, there is a problem in that the guardian might over-interpret the adolescent's behavior. Although various applications and webpages with such surveys have appeared, since such applications and webpages lack comprehensive judgements of psychiatrists, it may be difficult to get a professional judgement about a mental health state.

The discussions in this section are only to provide background information and do not constitute an admission of prior art.

### SUMMARY

The present disclosure is to assist a diagnosis of a psychiatrist by analyzing, through a means of a machine learning model, answers to survey questions that an adolescent may easily answer by their self through a mobile device or a digital device, predicting a mental health state of the adolescent or the possibility of an occurrence of a symptom of a mental illness and presenting it to a guardian or a psychiatrist.

An electronic device according to an embodiment, to assess a mental health state of an adolescent by using a survey formed based on an artificial intelligence, may comprise a question collecting module to collect physical information of a user through a pre-installed application, to verify survey questions regarding mental health depending on the physical information of the user, and to verify answers to the verified survey questions inputted by the user through the application; an artificial intelligence (AI) model module to form additional survey questions for the user after having verified the answers and to calculate a prediction rate of an appearance of symptoms of a mental illness regarding the additional survey questions by using AI models; a branching recommending module to verify a set of survey questions including a plurality of sub-questions depending on the calculated prediction rate of an appearance of symptoms of a mental illness; a question transmitting module to output the verified set of survey questions through the application; an object determining module to output result data by adjusting the prediction rate of an appearance of symptoms of a mental illness when the set of survey questions is not verified in the branching recommending module; and a result module to transmit the outputted result data.

In an electronic device according to an embodiment of the present disclosure, a method for assessing a mental health state of an adolescent by using a survey formed based on an artificial intelligence comprises: an operation of collecting physical information of a user through a pre-installed application, verifying survey questions regarding mental health depending on the physical information of the user, and verifying answers to the verified survey questions inputted by the user through the application; an operation of forming additional survey questions for the user after having verified the answers and calculating a prediction rate of an appearance of symptoms of a mental illness regarding the additional survey questions by using AI models; an operation of verifying a set of survey questions including a plurality of sub-questions depending on the calculated prediction rate of an appearance of symptoms of a mental illness; an operation of outputting the verified set of survey questions through the application; an operation of outputting result data by adjusting the prediction rate of an appearance of symptoms of a mental illness when the set of survey questions is not verified; and an operation of transmitting the outputted result data.

An electronic device for assessing a mental health state of an adolescent by using a survey formed based on an artificial intelligence and a method therefor have an effect to predict a mental health state of the adolescent and the possibility of an occurrence of a symptom of a mental illness by forming, by means of a machine learning model, survey questions to which the adolescent may easily answer by themselves and analyzing received answers.

Effects of the present disclosure are not limited to this. Various effects will be described below with reference to respective embodiments.

### BRIEF DESCRIPTION OF THE DRAWINGS

In order that the disclosure may be well understood, there will now be described various forms thereof, given by way of example, reference being made to the accompanying drawings, in which:

FIG. 1 is a block diagram illustrating a configuration of an electronic device for assessing a mental health state of a user by using a survey based on an artificial intelligence (AI) according to an embodiment of the present disclosure;

FIG. 2 is a block diagram illustrating a configuration of a question collecting module according to an embodiment of the present disclosure;

FIG. 3 is a block diagram illustrating a configuration of an artificial intelligence (AI) model module according to an embodiment of the present disclosure;

FIG. 4 is a block diagram illustrating a configuration of a branching recommending module according to an embodiment of the present disclosure;

FIG. 5 is a block diagram illustrating a configuration of an object determining module according to an embodiment of the present disclosure;

FIG. 6 is a block diagram illustrating a configuration of a result module according to an embodiment of the present disclosure;

FIG. 7 is a diagram showing a data set formed by collecting physical information of a user and available for training according to an embodiment of the present disclosure;

FIG. 8 is a diagram showing a result of a survey in an early stage made as data according to an embodiment of the present disclosure;

FIG. 9 is a diagram showing optimum survey questions formed by using AI models according to an embodiment of the present disclosure;

FIG. 10 is a graph showing results of trainings for performance of AI models by using data of entire questions according to an embodiment of the present disclosure;

FIG. 11 is a graph showing performance of AI models re-trained by using data of optimum survey questions formed depending on AI models according to an embodiment of the present disclosure;

FIGS. 12A and 12B are a diagram showing a decision tree model in which prediction values are branched into multiple AI models according to an embodiment of the present disclosure;

FIG. 13 is a graph showing results of verification of performance of trained AI models according to an embodiment of the present disclosure; and

FIG. 14 is a graph for determining an object for a periodic management by using prediction values of a plurality of AI models according to an embodiment of the present disclosure.

### DETAILED DESCRIPTION OF EMBODIMENTS

Since a technology described below may have various modifications and various embodiments, specific embodiments will be illustrated in the accompanying figures and described in detail. However, it should be understood that the below descriptions include all modifications, equivalents or substitutes which belong to the ideas and the scope of the described technology.

Terms, such as ‘first’, ‘second’, ‘A’, ‘B’ or the like may be used to describe various components. However, these terms do not limit the components, but are used only for distinguishing one component from another. For example, without being out of the scope of the right of the below-described technology, a first component may be referred to as a second component and, in a similar way, a second component may be referred to as a first component. The term ‘and/or’ means a combination of the relevant descriptions or any one of the relevant descriptions. For example, the description ‘A and/or B’ may be interpreted as ‘at least one of A and B’. In addition, the mark ‘/’ may be interpreted as ‘and’ or ‘or’.

In this specification, a term in a singular form may also mean a term in a plural form as long as there is no particular indication. It should be understood that the term ‘comprise’, etc. means the existence of the described characteristics, numbers, steps, operations, components, parts or their combinations, but does not mean the exclusion of the existence or the possibility of addition of one or more other characteristics, numbers, steps, operations, components, parts or their combinations.

Before describing the drawings in detail, it should be clarified that the classification of the components in this specification is made only by their main functions. That is, it is possible that two or more below-described components may be combined as one component or one component may be divided into two or more components depending on their detailed functions. In addition, each of the below-described components may additionally perform, in addition to its own main functions, some or all functions that another component performs. Otherwise, some of the main functions of each component may also be exclusively performed by another component.

In a method or an operational method, as long as any specific order is clearly indicated, steps for the method may be performed in an order different from the order in which the steps are described. That is, the steps may be performed in the order in which they are described, simultaneously, or in a reverse order.

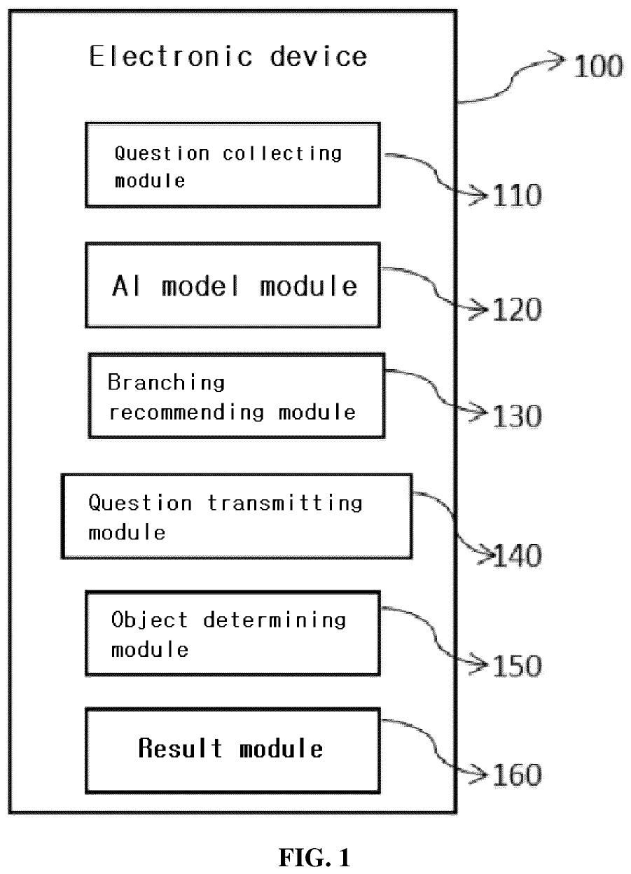

FIG. 1 is a block diagram illustrating a configuration of an electronic device for assessing a mental health state of a user by using a survey based on an AI according to an embodiment of the present disclosure.

Referring to FIG. 1, an electronic device 100 may comprise a question collecting module 110, an artificial intelligence (AI) model module 120, a branching recommending module 130, a question transmitting module 140, an object determining module 150, and a result module 160. In addition to them, other various components for collecting items for a survey based on an AI and assessing a mental health state based on answers of a user may be comprised.

According to an embodiment of the present disclosure, the electronic device 100 may assess a mental health state of a user and predict the possibility of an appearance of symptoms of a mental illness when the user inputs answers to questions for a survey by using an application comprising a technology of the subject disclosure. In addition, the electronic device 100 may transmit the answers of the user to an external server so that the server may perform an assessment of a mental health state of the user and a prediction of the possibility of an appearance of symptoms of a mental illness of the user.

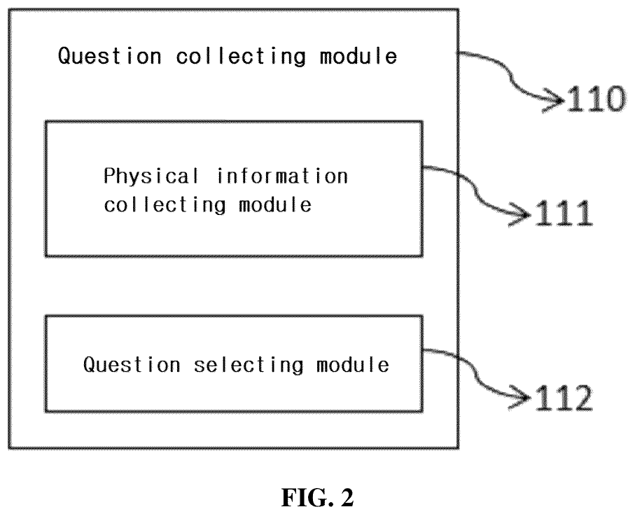

According to an embodiment of the present disclosure, the question collecting module 110 may control optimum questions for the survey based on physical information of the user collected by the application to be automatically presented to support the user's answers to the questions of the survey. A more specific description of the question collecting module 110 will be made below with reference to FIG. 2.

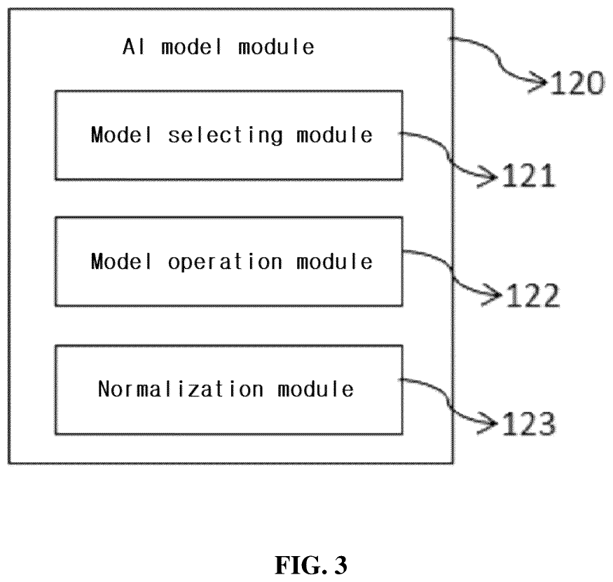

According to an embodiment of the present disclosure, the AI model module 120 may perform an operation allowing the prediction by forming data for inference regarding an optimum survey or additional questions, given to a user by a sub-question assigning module, to which answers are completed and being branched into a plurality of trained AI models to distinguish a symptom group from a normal group. More specific description of the AI model module 120 will be made below with reference to FIG. 3.

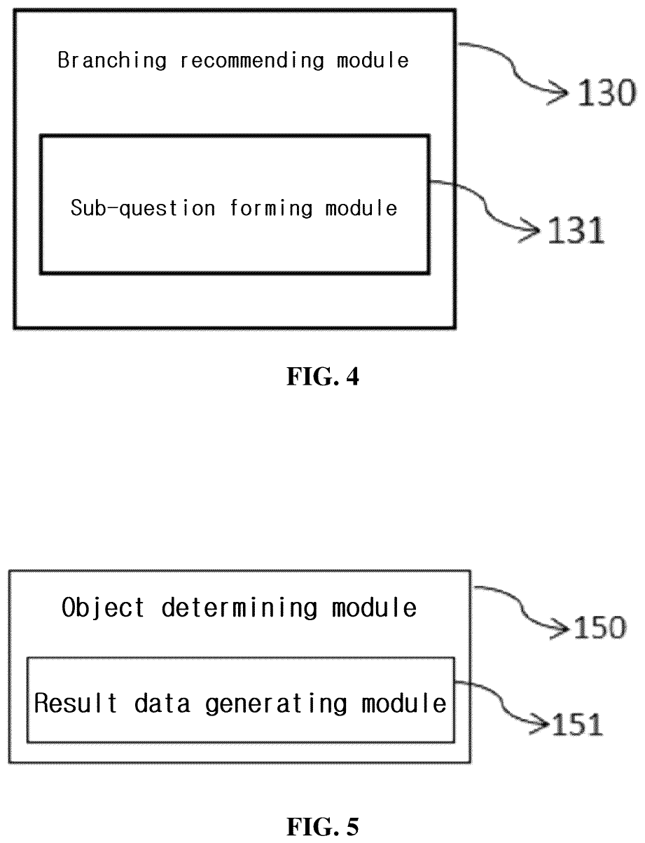

According to an embodiment of the present disclosure, when a prediction rate of a symptom appearance is equal to or higher than a reference value based on a prediction probability calculated in the AI model module 120, the branching recommending module 130 may search for sets of survey questions comprising more sub-questions for enhancing reliability of a model. More specific description of the branching recommending module 130 will be made below with reference to FIG. 4.

According to an embodiment of the present disclosure, the question transmitting module 140 may transmit sets of additional survey questions outputted by the branching recommending module 130 to the user through the application. For example, the question transmitting module 140 may provide the latest information to the user by using additional questions outputted by the branching recommending module 130.

According to an embodiment of the present disclosure, the question transmitting module 140 may secure reliability of additional questions by deriving from the user determinations easily influenced by the latest information and transmit notifications through the application so that the user may periodically use the application. As such, the question transmitting module 140 may enhance the prediction rate of the AI model by using answers to the additional questions presented to the user.

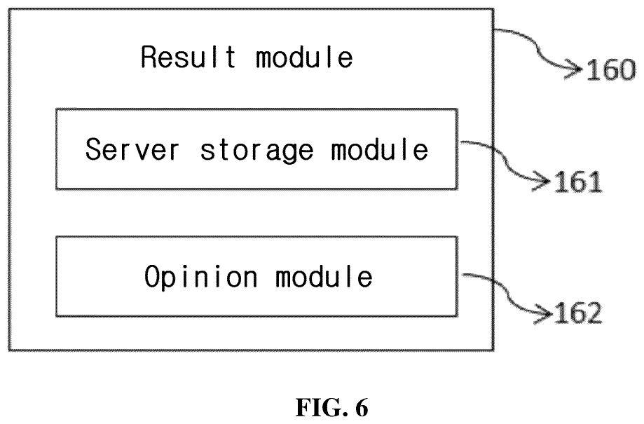

According to an embodiment of the present disclosure, the object determining module 150 may determine an adjusted prediction value based a threshold value indicating that there is no further model to be branched and output result data based on the number of branches of models. More specific description of the object determining module 150 will be made below with reference to FIG. 5.

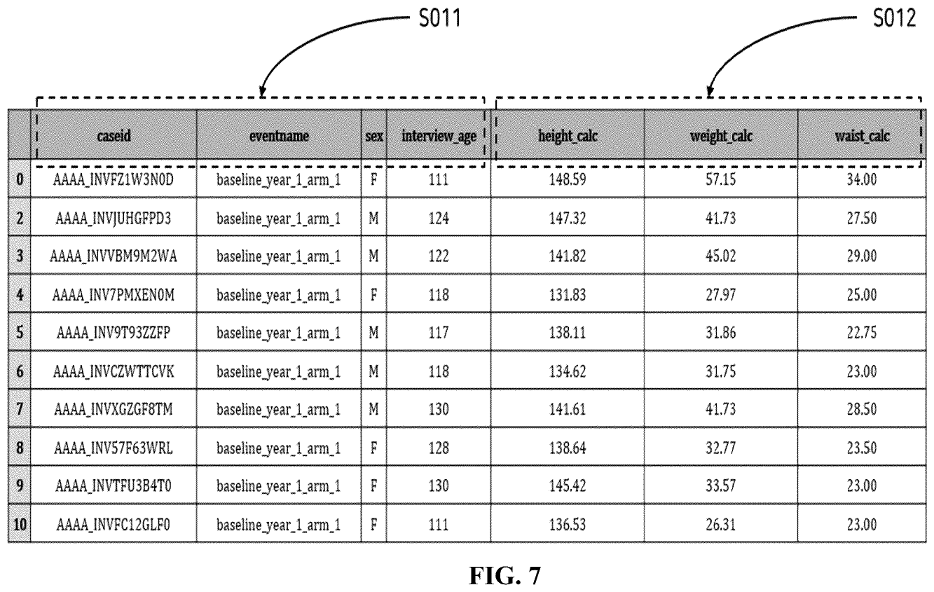

According to an embodiment of the present disclosure, the result module 160 may transmit to a psychiatrist a comprehensive result including the answers to the survey questions, the number of branches of models, classifications of the survey, or the like by using result data produced by the object determining module 150. More specific description of the result module 160 will be made below with reference to FIG. 6.

FIG. 2 is a block diagram illustrating a configuration of a question collecting module according to an embodiment of the present disclosure.

Referring to FIG. 2, the question collecting module 110 may comprise a physical information collecting module 111 and a question selecting module 112.

According to an embodiment of the present disclosure, the question collecting module 110 may induce a user to quickly answer a survey by collecting physical information of the user through an application, to which an AI model is applied, and presenting to the user optimum survey questions classified by using collected physical information of the user. In addition, the question collecting module 110 may generate a data set for the user comprising the optimum questions for deriving a primary effect.

According to an embodiment of the present disclosure, the physical information collecting module 111 may ask various questions regarding physical information of the user, such as age, height, weight, waist measurement, etc. and receive answers from the user. For example, the physical information collecting module 111 may collect physical information of the user based on the answers received from the user.

According to an embodiment of the present disclosure, the question selecting module 112 may present optimum survey questions selected by the AI model based on the physical information of the user collected by the physical information collecting module 111. For example, the question selecting module 112 may present optimum survey questions based on the physical information of the user so as to obtain reliable answers within a short time.

In a general survey in which answers to all the questions are required, the more questions there are, the more time it takes for a user to answer. In addition, the reliability of the user's answers may also decrease. Further, in a case when it is the first time for a user to use an application for a survey, it is highly likely that the brain of the user negatively perceives the application. On the other hand, according to the present disclosure, since the question selecting module 112 presents to a user optimum survey questions based on physical information of the user, the user can answer a relatively small number of questions and questions based on the physical information of the user's own. This leads to a reduced time for the answers and an enhanced reliability of the answers.

FIG. 3 is a block diagram illustrating a configuration of an AI model module according to an embodiment of the present disclosure.

Referring to FIG. 3, the AI model module 120 may comprise a model selecting module 121, a model operation module 122, and a normalization module 123.

According to an embodiment of the present disclosure, the AI model module 120 may predict an appearance of symptoms of a mental illness of the user by using a user data set generated by the question collecting module 110.

According to an embodiment of the present disclosure, the model selecting module 121 may select an AI model for prediction depending on the number of questions used for the survey. For example, the AI model may be a machine learning model such as Logistic Regression, Decision Tree, Random Forest, XGBoost, LightGBM or the like.

According to an embodiment of the present disclosure, the model operation module 122 may calculate a prediction value of an AI model selected by the model selecting module 121. For example, the model operation module 122 may control a plurality of AI models to perform predictions about the questions answered by the user and to perform final predictions by using weightings for prediction values set for respective AI models. In addition, the model operation module 122 may multiply the weightings set for the respective AI models and the prediction values to obtain adjusted prediction values and operate final prediction values by comparing these values.

According to an embodiment of the present disclosure, the model operation module 122 may generate a new feature by means of equation 1 using prediction values of multiple AI models and entropies of leaf nodes. For example, the model operation module 122 may generate a new feature by calculating adjusted prediction values of the respective models through the multiplication of the prediction values and the entropies or the weightings and adding up the calculated values.

Equation 1 is to generate a new feature by using the prediction values of the multiple AI models and the entropies of the leaf nodes according to an embodiment of the present disclosure.

$${F}_{new} = {\sum }_{i = 1}^{N} ( {P}_{i} ) \\ {P}_{i} = {p}_{i} {W}_{i} \qquad [ Equation 1 ]$$

According to an embodiment of the present disclosure, F_{new} is a new feature generated by equation 1. N is a total number of a plurality of AI models presented in the present disclosure. Pᵢ is an adjusted prediction value of each AI model, which is based on a prediction value and a weighting, and pᵢ is a prediction value of each AI model. Wᵢ means an entropy in a decision tree model and a weighting in other models.

According to an embodiment of the present disclosure, the normalization module 123 may verify the AI models in their prediction reliabilities based on final prediction values outputted from the model operation module 122 by using 7 performance evaluation indexes: receiver operating characteristics curve, accuracy, sensitivity, specificity, precision, recall, and F1 score. In addition, the normalization module 123 may also indicate evaluation indexes of a psychiatrist for prediction models in numbers.

According to an embodiment of the present disclosure, the evaluation indexes of a psychiatrist may be levels of prediction accuracy of AI models represented by numbers by a psychiatrist evaluating symptom predictions of AI models based on data used for trainings through the comparison with symptoms and diagnoses data obtained by using a screening tool such as the Kiddle Schedule for Affective Disorders and Schizophrenia Present and Lifetime Version (K-SADS-PL).

FIG. 4 is a block diagram illustrating a configuration of a branching recommending module according to an embodiment of the present disclosure.

Referring to FIG. 4, the branching recommending module 130 may comprise a sub-question forming module 131.

According to an embodiment of the present disclosure, the branching recommending module 130 may determine whether sub-questions need to be formed based on prediction values of the possibility of an appearance of symptoms of a mental illness outputted from the AI model module 120 and generate a new set of survey questions by selecting additional questions. For example, the branching recommending module 130 may construct, based on classification of survey questions, an AI model trained by entire questions and a plurality of AI models trained by a reduced number of survey questions including only questions important in respective groups.

According to an embodiment of the present disclosure, the sub-question forming module 131 may generate additional questions by using the constructed AI models and collect only answers to the additional questions as new data by comparing the generated additional questions with the questions to which the user has already answered.

Equation 2 is for a calculation reflecting feature importance values in a plurality of AI models.

$$SI = \{ {IMP}_{f 1} , {IMP}_{f 2} , … , {IMP}_{fN} \} \\ {IMP}_{fN} = \vert \frac{( {IMP}_{AF 1} + {IMP}_{Bf 1} + {IMP}_{Cf 1} + ⋯ + {IMP}_{Mf 1} )}{{N}_{models}} \vert \qquad [ Equation 2 ]$$

According to an embodiment of the present disclosure, SI is a set of collected feature importance values. IMP is an importance value. N is a number of a feature. A-M are identification information of AI models A to M. IMP_{IN} is a final importance value of one feature derived from a plurality of AI models. IMP^{Mf1} is an importance value of a first feature of a model. N_{models} is the number of AI models.

According to an embodiment of the present disclosure, an importance value of a first feature of each AI model may be represented in a form of IMP_{Mf1} and the branching recommending module 130 may calculate an average importance value for the first features by adding up the importance values for the first features of all the models and dividing the sum by the number of the models. The branching recommending module 130 may form a set by repeating the operation for calculating an average importance value as many times as the number of the entire features.

According to an embodiment of the present disclosure, the branching recommending module 130 may output additional questions, generated based on equation 2, to a user in an order of importance values. For example, the electronic device 100 may comprise an output device (not shown) for outputting additional questions in various forms of data (for example, sound, image, etc.) and the branching recommending module 130 may output additional questions through the output device.

FIG. 5 is a block diagram illustrating a configuration of an object determining module according to an embodiment of the present disclosure.

Referring to FIG. 5, the object determining module 150 may comprise a result data generating module 151.

According to an embodiment of the present disclosure, the object determining module 150 may determine, based on a prediction result outputted from the branching recommending module 130, a user as an object for management by annual periods or monthly periods. For example, the object determining module 150 may verify the possibility of an appearance of symptoms of a mental illness by using prediction values after the formation of the additional questions and determine a user, having a prediction value higher than a predetermined threshold value, as an object for management by periods.

According to an embodiment of the present disclosure, the result data generating module 151 may output result data to support the determination of a psychiatrist by providing answers to survey questions used by including a variable of an object for periodic management and prediction values of a plurality of models.

FIG. 6 is a block diagram illustrating a configuration of a result module according to an embodiment of the present disclosure.

Referring to FIG. 6, the result module 160 may comprise a server storage module 161 and an opinion module 162.

According to an embodiment of the present disclosure, the result module 160 may store result data outputted from the object determining module 150 in a database and transmit an opinion of a psychiatrist as data in a form for an application or a web service to a user's electronic device.

According to an embodiment of the present disclosure, the server storage module 161 may store result data in a database server in a form of a cloud or on-premise solution in the manner of de-identification.

According to an embodiment of the present disclosure, when the result data has completed to be stored, the opinion module 162 may generate data in a form for an application or a web service with the stored result data so that a psychiatrist may refer thereto. Then, the psychiatrist may transfer their opinion in a form of data for an application or a web service to a user.

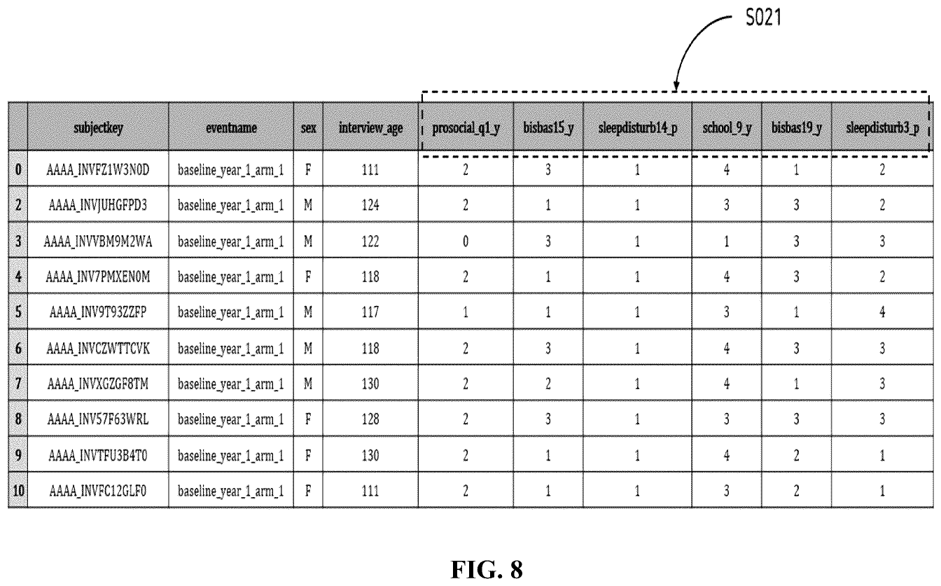

FIG. 7 is a diagram showing a data set formed by collecting physical information of a user and available for training according to an embodiment of the present disclosure.

Referring to FIG. 7, the question collecting module 110 may collect physical information of a user by using the physical information collecting module 111 and verify survey questions based on the collected physical information by using the question selecting module 112.

According to an embodiment of the present disclosure, the question collecting module 110 may verify and store, by unique identifiers (caseid) of users used inside a system, collected physical information such as times of a survey of users (event name), the sexes of users (sex), the monthly ages of users (interview_age) as indicated by S011.

According to an embodiment of the present disclosure, the question collecting module 110 may verify and store, as physical information for the unique identifiers, heights of users (height_calc), weights of users (weight_calc), and waist measurements of users (waist_calc) as indicated by S012.

According to an embodiment of the present disclosure, the question collecting module 110 may perform the above described operations indicated by S011 and S012 based on information inputted by users through an application or a web page.

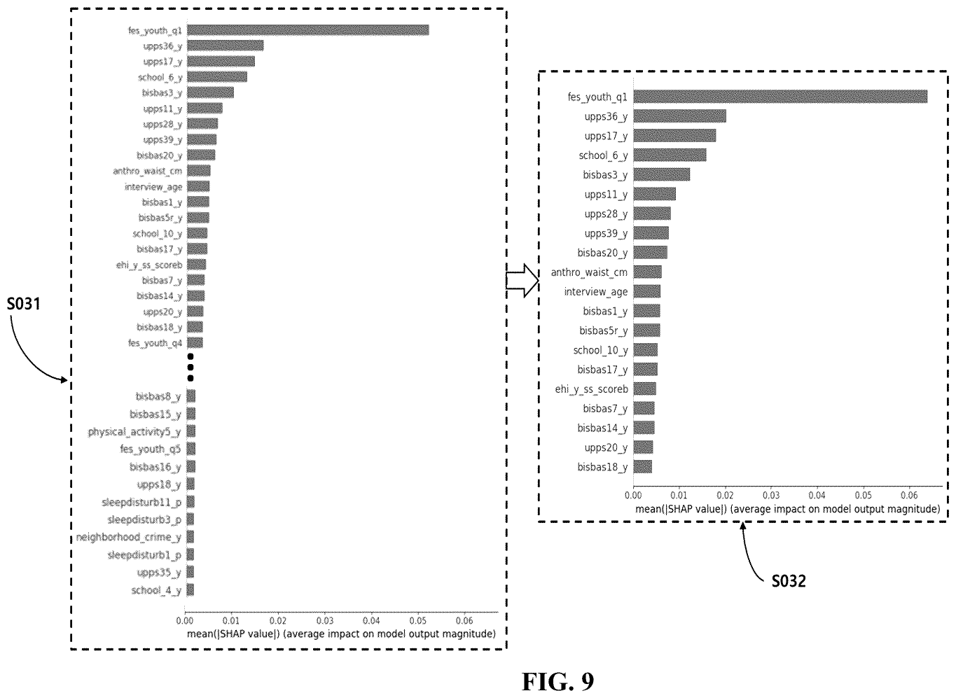

FIG. 8 is a diagram showing a result of a survey in an early stage made as data according to an embodiment of the present disclosure.

Referring to FIG. 8, the question collecting module 110 may verify optimum survey questions selected by AI models at an early stage of a service use and transmit them to a user.

According to an embodiment of the present disclosure, the question collecting module 110 may verify and store scores regarding respective questions, which are a question regarding pro-sociality (prosocial), a question regarding a behavior inhibition system (bisbas), a question regarding a behavioral activation system, a question regarding a sleep disturbance scale (sleep disturb), and a question regarding a school life (school) as indicated by S021, based on the physical information of the user.

According to an embodiment of the present disclosure, the question collecting module 110 may verify values inputted in items of a time of a survey of a user (eventname), the sex of a user (sex), and the monthly age of a user (interview_age) and assign values for answer data regarding respective questions based on score sections predetermined to correspond to the respective input values.

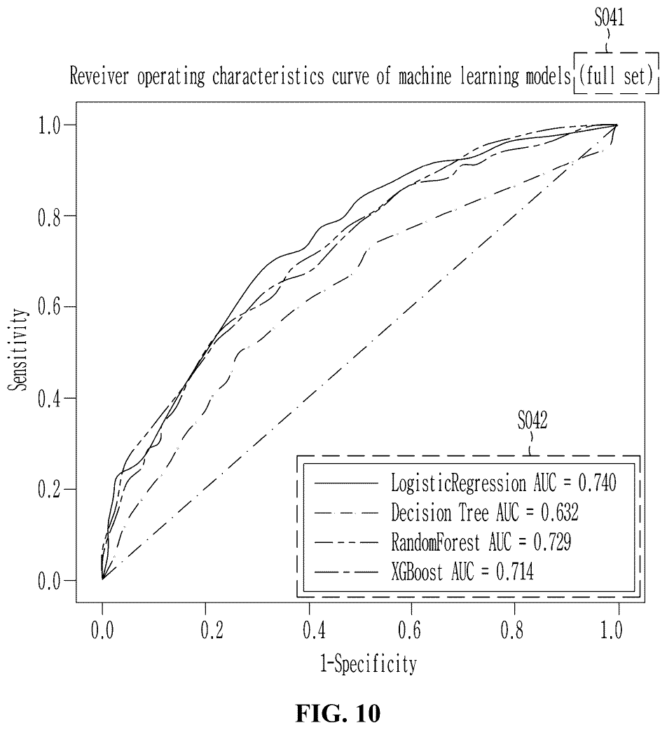

FIG. 9 is a diagram showing optimum survey questions formed by using AI models according to an embodiment of the present disclosure.

Referring to FIG. 9, the question collecting module 110 may verify questions to be important features for prediction by using a plurality of AI models, arrange the questions in a descending order based on importance values of the respective verified questions, and verify features of high-ranked questions within a reference range.

According to an embodiment of the present disclosure, the question collecting module 110 may determine importance of each of all the features based on a plurality of AI models as shown in S031. For example, the question collecting module 110 may determine high-ranked features within a range, in which the performance decrease of an AI model is minimized, based on the importance of each of all the features determined above.

According to an embodiment of the present disclosure, the question collecting module 110 may form survey questions including the high-ranked features determined above as shown in S032 and transmit them to a user. For example, the question collecting module 110 may form a number of survey questions, in which the number is predetermined for a corresponding user, and an optimum number may be set based on information related to a user.

According to an embodiment of the present disclosure, since the question collecting module 110 forms survey questions including high-ranked features, the number of survey questions may be reduced from 104 in total to 20.

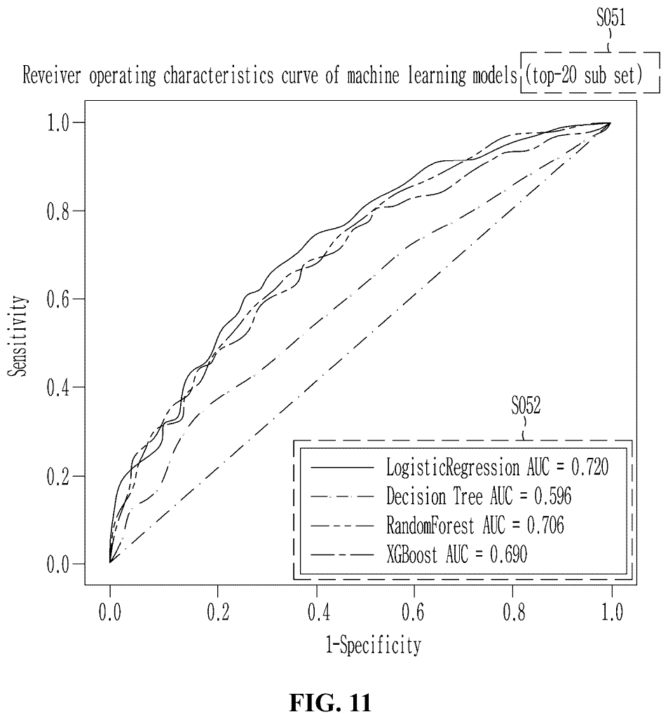

FIG. 10 is a graph showing results of trainings for performance of AI models by using data of entire questions according to an embodiment of the present disclosure.

Referring to FIG. 10, the AI model module 120 may train AI models with collected data of users, verify the data, separate a test set of data from the verified data, and test performance of the AI models with the test set. For example, the AI model module 120 may evaluate the performance of the AI models with evaluation indexes of specificity and sensibility and verify evaluation values for the respective evaluation indexes.

According to an embodiment of the present disclosure, the AI model module 120 may output entire survey questions by using a plurality of AI models S042 and show the performances of the AI models regarding the outputted entire survey questions represented by a graph S041 with evaluation values for the specificity and the sensibility.

According to an embodiment of the present disclosure, although 4 types of AI models as a plurality of AI models S042 are represented in the figure, the AI model module 120 may set the number of AI models to be used depending on physical information of users or the numbers of survey questions for users.

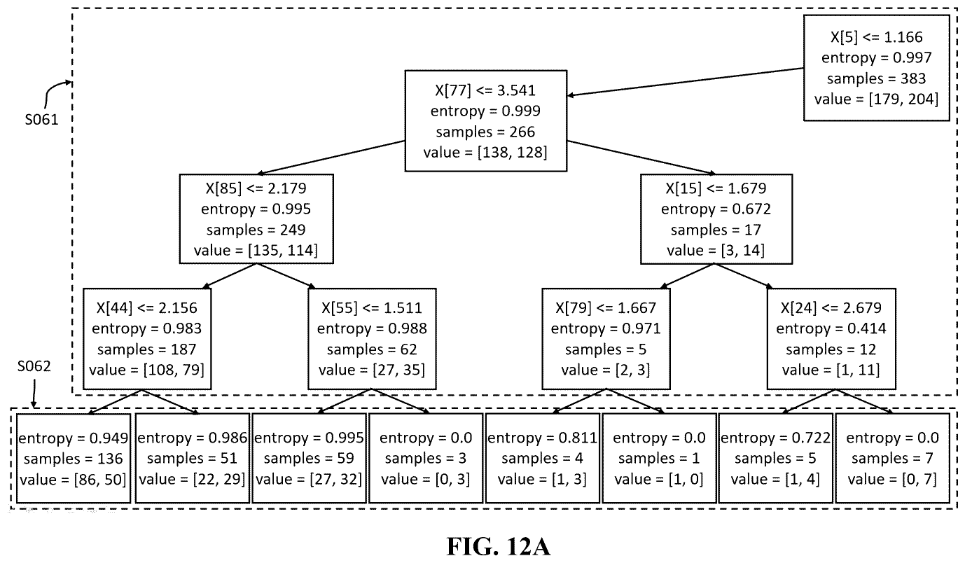

FIG. 11 is a graph showing performance of AI models re-trained by using data of optimum survey questions formed depending on AI models according to an embodiment of the present disclosure.

Referring to FIG. 11, the AI model module 120 may form a data set (top-20 sub set) with the collected optimum survey questions as a test set, evaluate performances of AI models by using the test set, and represent the performances in a graph S051. For example, the AI model module 120 may show the performances of the AI models regarding the optimum survey questions in the graph S051.

According to an embodiment of the present disclosure, the AI model module 120 may determine performances of a plurality of AI models S052 including the 4 types of AI models regarding the optimum survey questions.

According to an embodiment of the present disclosure, the AI model module 120 may verify a performance decrease rate by comparing the graph for the performances regarding the optimum survey questions S051 with the graph for the performances regarding the entire survey questions S041. When the performance decrease rate of the AI models is lower than a number decrease rate (for example, 80.77%) through comparison of the number of the optimum survey questions (for example, 20) with the number of the entire survey questions (for example, 104), the AI model module 120 may determine that the performance decrease rate of the AI models is within a reference range, and thus, determine the optimum survey questions as final survey questions.

According to an embodiment of the present disclosure, since the electronic device 100 may let a user answer optimum survey questions of which the performance decrease rate of the AI models is within a reference range, the user may complete answering within 10 minutes or so, whereas it would generally take about 50 minutes for a user to answer the entire survey questions off-line.

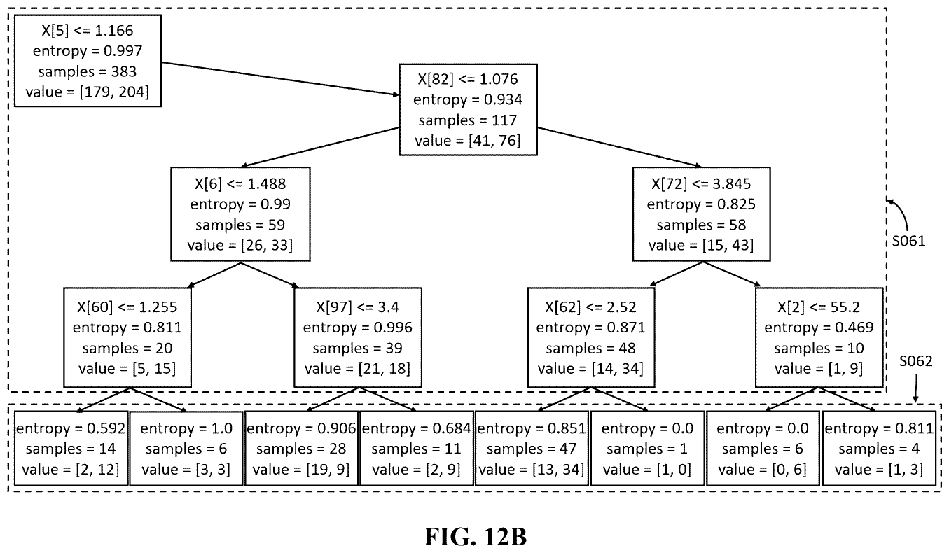

FIGS. 12A and 12B are a diagram showing a decision tree model in which prediction values are branched in forms of AI models according to an embodiment of the present disclosure.

Referring to FIGS. 12A and 12B, the decision tree model may comprise an optimum tree S061 without leaf nodes and leaf nodes S062.

According to an embodiment of the present disclosure, the AI model module 120 may adjust a prediction value by using a prediction value of an AI model and additionally using entropy of a final node corresponding to the AI model.

According to an embodiment of the present disclosure, the AI model module 120 may deduce a path of answer data used for prediction by using the optimum tree S061 and the leaf nodes S062 and verify a leaf node corresponding to the path to adjust a prediction value with the relevant entropy.

According to an embodiment of the present disclosure, the AI model module 120 may make prediction values for answer data of a user and entropies as data to transmit them to the branching recommending module 130.

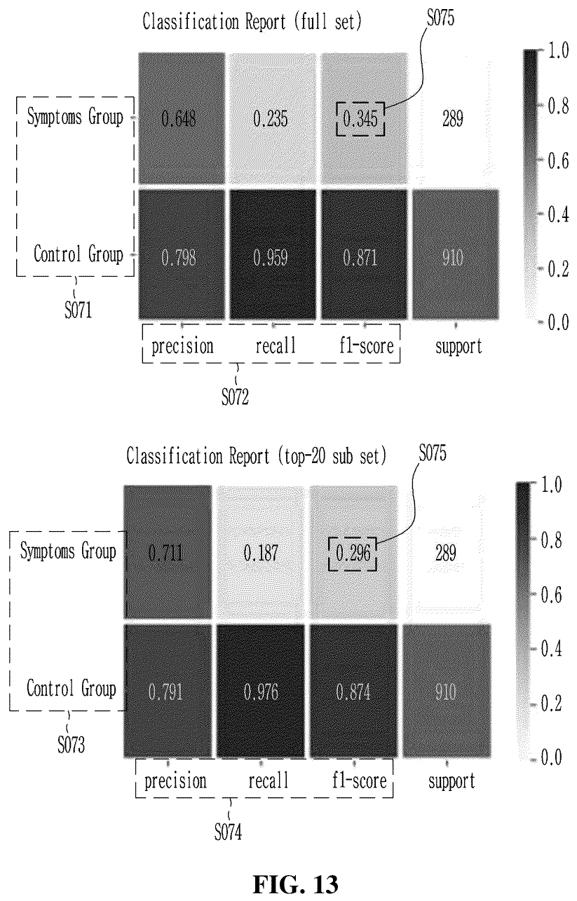

FIG. 13 is a graph showing results of verification of performances of trained AI models according to an embodiment of the present disclosure.

FIG. 13 shows graphs of classification results (classification report) regarding the entire survey questions (full set) and reduced survey questions (top-20 sub set), which are the optimum survey questions.

According to an embodiment of the present disclosure, in each classification result, the AI model module 120 may make binary classification of a normal group and a symptoms group S071, S073 and each group of users may be classified into several groups based on a performance verification method such as precision, recall, and an f1-score S072, S074.

According to an embodiment of the present disclosure, the AI model module 120 may verify values of the f1-scores S075 of the symptoms groups as indexes for prediction. For example, the AI model module 120 may verify that, although an AI model for the reduced survey questions has a lower f1-score than a f1-score of an AI model for the entire survey questions, it has a higher precision value. In this case, the AI model module 120 may induce a more precise prediction of the AI model for the reduced survey questions by collecting some additional questions.

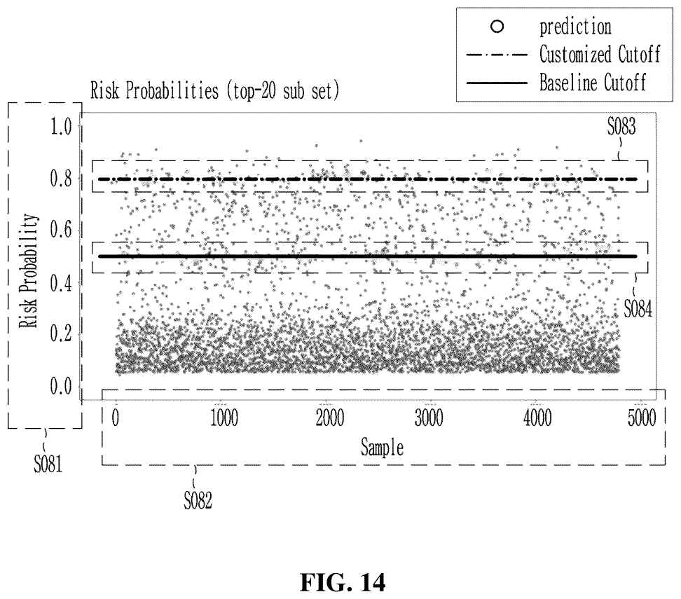

FIG. 14 is a graph for determining an object for a periodic management by using prediction values of a plurality of AI models according to an embodiment of the present disclosure.

Referring to FIG. 14, the object determining module 150 may represent a risk probability of symptoms S081 based on the number of pieces of data of a user S082 in a graph. In the graph, for example, 0.0 may mean 0% and 1.0 may mean 100%.

According to an embodiment of the present disclosure, the object determining module 150 may adjust a threshold point according to an opinion of a psychiatrist and indicate the adjusted threshold point S083 in the graph. In addition, the object determining module 150 may verify a basic determination threshold point S084 of the AI models and indicate the determination threshold point S084 in the graph.

According to an embodiment of the present disclosure, the object determining module 150 may indicate a prediction value for each user in the graph with a dot. When the sensitivity of an AI model exceeds the determination threshold point S084, the sensitivity may be set to be low by setting the adjusted threshold point S083 as the determination threshold point S084 according to a determination of a psychiatrist. Then, the object determining module 150 may periodically perform an operation of setting the determination threshold point S083 and the adjusted threshold point S083 and select an object for which a survey will be conducted in every corresponding period.

Embodiments according to the present disclosure may be implemented by various means, for example, hardware, firmware, software, or their combination. When it comes to hardware, an embodiment of the present disclosure may be implemented by one or more of application specific integrated circuits (ASICs), digital signal processors (DSPs), digital signal processing devices (DSPDs), programmable logic devices (PLDs), field programmable gate arrays (FPGAs), processors, controllers, micro controllers, microprocessors, and the like.

When it comes to firmware or software, an embodiment of the present disclosure may be implemented in forms of a module, a process, a function, or the like to perform the above-described functions or operations and written in recording media readable by various computer means.

Here, the recording media may include program instructions, data files, data structures, or the like separately or in combination. Program instructions to be written in the recording media may be specifically designed and constructed for the present disclosure or known to persons in the field of computer software to be available to them. For example, the recording media may comprise hardware devices specifically formed to store and execute program instructions, such as magnetic media like hard disks, floppy disks, and magnetic tapes; optical media like compact disk read only memories (CD-ROMs) and digital video disks (DVDs); magneto-optical media like floptical disks, ROMs, RAMs, flash memories, or the like. Examples for program instructions may comprise machine language codes formed by, for example, compilers and high-level language codes executable by computers by using interpreters as well. Hardware devices as described above may be configured to operate as one or more software modules in order to perform the operations of the present disclosure, and vice versa.

In addition, a device or a terminal according to the present disclosure may be driven by instructions to cause one or more processers to execute the above-described functions and processes. For example, such instructions may comprise interpretable instructions like script instructions, such as JavaScript or ECMAScript instructions, executable codes, or other instructions stored in computer readable media. Further, a device according to the present disclosure may be implemented in a distributed form, such as Server Farm, over the network or implemented in a single computer device.

In addition, computer programs (also known as programs, software, software applications, or codes) to be installed in a device according to the present disclosure and to execute a method according to the present disclosure may be written by any types of programming languages including compiled or interpreted languages or innate or procedural languages and may be deployed in any forms including stand-alone programs, components, sub-routines or other units appropriate for being used in a computer environment. Computer programs do not necessarily correspond to files in the file system. A program may be stored in a single file provided to a requested program, in multiple interactive files (for example, files storing one or more modules, subprograms, or portions of code), or in a part of a file that holds other programs or data (for example, one or more scripts stored in a markup language document). A computer program may be deployed to be executed on multiple computers or on one computer, located at a single site or distributed across multiple sites and interconnected by a communications network.

Although respective figures are separately described for the convenience of description, it is also possible to design to implement a new embodiment by combining the embodiments described with respect to the figures. In addition, in the present disclosure, the configurations and methods of the above-described embodiments are not limitedly applied, but all or some of the respective embodiments may be selectively combined for various modifications.

Moreover, although preferred embodiments are illustrated and described in the above, the present disclosure is not limited to the above-described specific embodiments, but can be variously modified by a person skilled in the art, to which the present disclosure pertains, without being beyond the purport of the claims. In addition, such modified embodiments should not be understood to depart from the technological idea or the prospect of the present disclosure.

What is claimed is:

1. An electronic device for assessing a mental health of an adolescent by using a survey formed based on an artificial intelligence (AI), comprising:
a question collecting module to collect physical information of a user through a pre-installed application, to verify survey questions regarding a mental health state depending on the physical information of the user, and to verify answers to the verified survey questions inputted by the user through the application;
an artificial intelligence (AI) model module to form additional survey questions for the user after having verified the answers and to calculate a prediction rate of an appearance of symptoms of a mental illness regarding the additional survey questions by using AI models;
a branching recommending module to verify a set of survey questions including a plurality of sub-questions depending on the calculated prediction rate of an appearance of symptoms of a mental illness;
a question transmitting module to output the verified set of survey questions through the application;
an object determining module to output result data by adjusting the prediction rate of an appearance of symptoms of a mental illness when the set of survey questions is not verified in the branching recommending module; and
a result module to transmit the outputted result data.

2. The electronic device of claim 1, wherein the question collecting module comprises a physical information collecting module to output questions regarding age, height, weight, or waist measurement of the user through the application, to receive answers to the outputted questions to collect the answers as physical information of the user.

3. The electronic device of claim 2, wherein the question collecting module further comprises a question selecting module to output by using the AI models, among the questions regarding the physical information of the user, survey questions to which answers can be obtained within a predetermined time through the application.

4. The electronic device of claim 1, wherein the AI model module comprises:
a model selecting module to select an AI model depending on the number of additional survey questions;
a model operation module to verify a prediction value of the selected AI model regarding the additional survey questions by using the AI model selected in the model selecting module; and
a normalization module to verify the selected AI model according to evaluation indexes preset based on the verified prediction value.

5. The electronic device of claim 4, wherein the model operation module generates a new feature by means of equation 1, which is
$${F}_{new} = {\sum }_{i = 1}^{N} ( {P}_{i} ) \\ {P}_{i} = {p}_{i} {W}_{i}$$
wherein F_{new} is a new feature, N is a total number of a plurality of AI models, Pᵢ is an adjusted prediction value of each AI model, pᵢ is a prediction value of each AI model, and Wᵢ is an entropy in a decision tree model and a weighting in other AI models.

6. The electronic device of claim 5, wherein the model operation module outputs a set of feature importance values collected by the new features by means of equation 2, which is
$$SI = \{ {IMP}_{f 1} , {IMP}_{f 2} , … , {IMP}_{fN} \} \\ {IMP}_{fN} = \vert \frac{( {IMP}_{AF 1} + {IMP}_{Bf 1} + {IMP}_{Cf 1} + ⋯ + {IMP}_{Mf 1} )}{{N}_{models}} \vert$$
wherein SI is a set of collected feature importance values, IMP is an importance value, N is a number of a feature, A-M are identification information of AI models A to M, IMP_{IN} is a final importance value of one feature derived from a plurality of AI models, IMP_{Mf1} is importance values of first features of the AI models, and N_{models} is the number of AI models.

7. The electronic device of claim 6, wherein the AI model confirms survey questions, corresponding to the feature importance values included in the set of calculated feature importance values, as the additional survey questions.

8. The electronic device of claim 6, wherein the branching recommending module outputs through the application the additional survey questions confirmed by the AI model module in a descending order of feature importance values calculated by equation 2.

9. The electronic device of claim 1, wherein the object determining module periodically re-verifies a set of survey questions for the user when the prediction rate of an appearance of symptoms of a mental illness exceeds a predetermined value.

10. The electronic device of claim 1, wherein the result module converts the outputted result data into data in a form for the application or a web service and transmits it to a device of a predetermined psychiatrist.

11. A method for assessing a mental health state of an adolescent by using a survey formed based on an artificial intelligence (AI) comprising:
an operation of collecting physical information of a user through a pre-installed application, verifying survey questions regarding a mental health depending on the physical information of the user, and verifying answers to the verified survey questions inputted by the user through the application;
an operation of forming additional survey questions for the user after having verified the answers and calculating a prediction rate of an appearance of symptoms of a mental illness regarding the additional survey questions by using AI models;
an operation of verifying a set of survey questions including a plurality of sub-questions depending on the calculated prediction rate of an appearance of symptoms of a mental illness;
an operation of outputting the verified set of survey questions through the application;
an operation of outputting result data by adjusting the prediction rate of an appearance of symptoms of a mental illness when the set of survey questions is not verified; and
an operation of transmitting the outputted result data.

12. The method of claim 11, further comprising:
an operation of outputting questions regarding age, height, weight, or waist measurement of the user through the application and collecting the answers as physical information of the user by receiving answers to the outputted questions.

13. The method of claim 12, further comprising:
an operation of outputting by using the AI models, among the questions regarding the physical information of the user, survey questions to which answers can be obtained within a predetermined time through the application.

14. The method of claim 11, further comprising:
an operation of selecting an AI model depending on the number of additional survey questions;
an operation of verifying a prediction value of the selected AI model regarding the additional survey questions by using the selected AI model; and
an operation of verifying the selected AI model according to evaluation indexes preset based on the verified prediction value.

15. The method of claim 14, further comprising:
an operation of generating a new feature by means of equation 1, which is
$${F}_{new} = {\sum }_{i = 1}^{N} ( {P}_{i} ) \\ {P}_{i} = {p}_{i} {W}_{i}$$
wherein F_{new} is a new feature, N is a total number of a plurality of AI models, Pᵢ is an adjusted prediction value of each AI model, pᵢ is a prediction value of each AI model, and Wᵢ is an entropy in a decision tree model and a weighting in other AI models.

16. The method of claim 15, further comprising:
an operation of outputting a set of feature importance values collected by the new features by means of equation 2, which is
$$SI = \{ {IMP}_{f 1} , {IMP}_{f 2} , … , {IMP}_{fN} \} \\ {IMP}_{fN} = \vert \frac{( {IMP}_{AF 1} + {IMP}_{Bf 1} + {IMP}_{Cf 1} + ⋯ + {IMP}_{Mf 1} )}{{N}_{models}} \vert$$
wherein SI is a set of collected feature importance values, IMP is an importance value, N is a number of a feature, A-M are identification information of AI models A to M, IMP_{IN} is a final importance value of one feature derived from a plurality of AI models, IMPM is importance values of first features of the AI models, and N_{models} is the number of AI models.

17. The method of claim 16, further comprising:
an operation of confirming survey questions, corresponding to the feature importance values included in the set of calculated feature importance values, as the additional survey questions.

18. The method of claim 16, further comprising:
an operation of outputting through the application the additional questions confirmed by the AI model module in a descending order of feature importance values calculated by equation 2.

19. The method of claim 11, further comprising:
an operation of periodically re-verifying a set of survey questions for the user when the prediction rate of an appearance of symptoms of a mental illness exceeds a predetermined value.

20. The method of claim 11, further comprising:
an operation of converting the outputted result data into data in a form for the application or a web service and transmitting it to a device of a predetermined psychiatrist.
